(19) United States
(12) Patent Application Publication
(10) Pub. No.: US 2023/0349872 A1
(43) Pub. Date: Nov. 2, 2023

(54) GUN SHOT RESIDUE FIELD KIT

(71) Applicant: VERITEQUE USA, INC., Carson City, NV (US)

(72) Inventor: Christian Loane, Carson City, NV (US)

(21) Appl. No.: 18/006,011

(22) PCT Filed: Jul. 20, 2021
(86) PCT No.: PCT/US2021/042416

§ 371 Date: Jan. 19, 2023

Related U.S. Application Data
(60) Provisional application No. 63/053,758, filed on Jul. 20, 2020.
(60) Provisional application No. 63/106,180, filed on Oct. 27, 2020.

(51) Int. Cl. G01N 31/22; G01N 1/12; G01N 21/78

(52) U.S. Cl. CPC G01N 31/22; G01N 1/12; G01N 21/78; G01N 2001/028

(57) ABSTRACT

Provided herein, inter alia, a portable test device, mass manufacture methods thereof, and methods of use for presumptive identification of gunshot residues and/or explosive residues. The portable test device can be a kit including a colorimetric reagent(s), a solvent(s), and a swab(s). The colorimetric reagent(s) and solvents(s) and a swab(s) can be provided in hermetically sealed light, air and moisture proof packaging.

### CROSS-REFERENCE TO RELATED APPLICATIONS

This application claims the benefit of U.S. Provisional Patent Application No. 63/053,758, filed on Jul. 20, 2020, entitled “Gun Shot Residue Field Kit,” and U.S. Provisional Patent Application No. 63/106,180, filed on Oct. 27, 2020, entitled “GSR Field Kit.” The entirety of each of these applications is incorporated by reference.

### BACKGROUND

### Field

This disclosure generally relates to colorimetric Gun Shot Residue (GSR) detection methods and kits thereof.

Post blast gasses and burnt particulate is generated when a weapon's firing pin strikes the primer of a cartridge, which ignites the explosive primer compound, sending a flame into the cartridge case, which in turn ignites the gunpowder which subsequently burns, causing it to change from a solid to gas[1]. As the bullet exits the firearm, gasses and burnt fine particulates escape via any gap and opening in the weapon mechanism, including the barrel and breach. The residues are expelled from the barrel in a smoky cone shaped pattern[1].

The further gunshot residues travel, the broader and less concentrated the residue becomes. Because the various elements included in gunshot residues are very small and lack mass, they lose their energy rapidly, and subsequently do not travel far from the source of ignition[1].

Commercially available colorimetric GSR detection kits are available. For example, Fisher Scientific®, Carolina®, Sirchie®, Safariland®, Innovating Science®, Tri-Tech Forensics, Edu-Lab®, Sandia National Laboratories®, and many others, provide kits containing liquid reagent dropper bottles, powder reagents and alcohol swabs. Arrowhead Forensics, DetectaChem® and many others provide kits containing aerosolized liquid reagent spray cans or crushable glass ampoules filled with liquid reagents.

However, the aforementioned kits contain many un-desirable features. In one aspect, such kits can employ highly corrosive and hazardous liquids. In another aspect, such kits can contain glass bottles and ampoules which are bulky, cumbersome and break, causing sharps inflicting cuts, exposing skin to direct hazardous chemical contact. In a further aspect, such kits can contain aerosolized spray cans, which create chemical fume and spray drift of hazardous chemicals. In an additional aspect, such kits can employ bulky packaging and large waste stream, with limited recycle potential. In a further aspect, such kits are difficult to air-freight, which adds significant cost to end users. The aforementioned kits, during use, will leave residues of chemical reagents on surfaces and residues of forensic significance, thus contaminating crime scenes and destroying vital evidence.

The aforementioned kits generally have slow chemical reactivity, often taking 60 seconds or more for a single analysis. As a result, when many samples are tested at a crime scene, the required to complete analysis of all samples can be significant.

Thus, new methods for the detection of GSR are needed.

### SUMMARY

Provided herein, inter alia, are methods and kits for detecting Gun Shot Residue (GSR) and conventional and home-made explosive residue(s). The present disclosure relates to a portable test kit capable of identifying the presence of GSR and conventional and home-made explosive residue(s) on surfaces, a process to cheaply mass produce the portable test kit and achieve long term commercial shelf life (e.g., shelf life in the range of about 1 to about 5 years), and a method to use the portable test kit.

The present disclosure provides a design for a low cost, mass producible, field deployable, spot test kit which will facilitate identification of GSR and conventional and home-made explosive residue(s) from many surfaces, while minimizing operator exposure to hazardous chemicals. Examples of such surfaces, can include, but are not limited to, skin, metals, glass and fabrics.

It is a further objective of embodiments of the present disclosure to provide a kit with a very simple sample collection technique. Beneficially, the kit can minimize the destruction and contamination of surfaces, forensic residue, crimes scenes and the like, thus greatly aiding the forensic process.

It is a further objective of embodiments of the present disclosure to design a kit, including simple absorbent material (e.g., a cotton swab or fiber wipe(s)), with all color change reagents supplied in simple hermetically sealed closures, constructed of non-hazardous materials and components.

In other embodiments, the present disclosure provides for a design of a portable test kit, which has low cost, mass manufacture capability, in the order of millions of units per annum, while achieving commercial kit shelf life, in the order of several years (e.g., about 1 to about 5 years) and a reduced false alarm rate(s).

In embodiments, the portable test kit described herein includes an absorbent material (e.g., a cotton swab or fiber wipe(s)) that is pre-wetted with a solvent and a dry powder reagent. The dry powder reagent is provided in any suitable modality for end use which is simple to mass produce and achieves long term shelf life, ease in shipment and carriage and simplicity in use and disposal. By way of example, and without limitation, such modalities may include dry powder stored within a container, dry powder affixed to a substrate, or dry powder compressed into a free-standing form factor. Examples of containers can include, but are not limited to, sachets, ampoules, vials, or blister packs. Examples of substrates can include, but are not limited to, cards or strips formed from suitable materials such as paper or plastic). Examples of free-standing form factors include pressed sheets and pressed tablets.

In other aspects, embodiments of the present disclosure provide for methods of kit manufacture and use.

In other embodiments, provided herein is a portable detection kit for identifying the presence of GSR and conventional and home-made explosive residue(s). In embodiments, the process for producing said kit and method of using said kit are provided herein. In embodiments, the kit, without limitation includes a dry chemical reagent(s), and an absorbent material (e.g., a cotton swab) pre-wetted with solvent(s).

In embodiments, the kit, without limitation may be provided for use in any combination as dry powder(s) and solvents(s) that are separated or mixed together in any combination as dissolved solution(s) or homogenized suspension(s). The kit can further store dry powder(s) solvent(s), solutions or suspensions in combination of packaging or containers or support articles. The kit and its components are provided in individual hermetically sealed light, air and moisture proof packaging for ease in transport, handling and long term storage prior to use.

The portable detection kit described herein can be capable of detecting target GSR and/or conventional and home-made explosive residue(s) in a variety of forms. Examples can include solid particulates (e.g., particulates created from the ignition of cartridge primer explosives and subsequent burning of gun powder, producing a phase change from solid to hot gas, cooling of the discharged gasses, thus forming, a deposition of particulate residue(s)) and pre-cursors to manufacture of and pre- and post-blast residues of conventional and home-made explosive residue(s).

Also provided herein are methods of preparing the portable detection kit described herein. In embodiments, the dry chemical reagents can be colorimetric reagents that, when combined with a solvent(s), undergo physico-chemical interaction with the GSR and conventional and home-made explosive residue(s) to produce a colored reaction product, resulting in a visible color change also known as a presumptive colorimetric indication.

Embodiments of the dry chemical reagent(s) without limitation may be selected from the group consisting of (a) dyes (e.g. azo, formazan, mordant), (b) cyclic enols, (c) carbamates, (d) sulphonic acids, (e) imidazolines, (f) thio compounds, (g) diazotising reagents, (h) oximes, (i) phenylamines, (j) phenanthrolines, (k) hydrazides, (l) arsonium, (m) boron based compounds, (n) alkaline silicates, (o) hydroxide salts, (p) nucleophiles, or (q) metal salts. Each of these may be used for the detection of one or more of the components of GSR and conventional and home-made explosive residue(s), including but not limited to, oxides of nitrogen, including aromatic, aliphatic or inorganic or metal particles.

In embodiments, GSR or conventional or home-made explosive residue(s) (ER) may be detected in molecular and ionic form as (i) nitroaromatics, (ii) nitrites, (iii) nitrates, (iv) nitroamines, (v) nitroesters, (vi) metal particulate (vii) powerful oxidizing agents (oxidizers) by using any combination(s) of the above-discussed dry chemical reagents and an absorbent material pre-wetted with a solvent (e.g., a cotton swab) to facilitate a presumptive colorimetric indication, thus identifying the presence of GSR or conventional or home-made explosive residue(s) (ER) in the form of (i)-(vii) discussed above.

Embodiments of the (i) nitroaromatics may be presumptively identified using any combination of an alkaline nucleophile chosen from dry chemical reagents (a)-(q) in combination with an absorbent material pre-wetted with an organic or aqueous solvent.

NITROAROMATICS—In an embodiment, the colorimetric reagent can be configured to detect nitroaromatics. Examples can include, but are not limited to, sodium silicate, sodium teraborate, hydroxide salts, N,N,dimethyl-1-naphthylamine, N-1-naphthyl-ethylene-diamine dihydrochloride, or sodium borohydride. Such colorimetric reagents facilitate presumptive identification of nitroaromatic compounds, via formation of a colored Meisenheimer complex, in any combination with water, dimethylsulfoxide (DMSO), alcohol(s), acetone, and the like. The nitroaromatic compound(s) may be present in the form of pre- and post-blast gun powder(s), conventional and home-made explosives, or pre-cursors to explosives and gun powders, which may or may not be used in the bullet constructions, weapons and/or Improvised Explosive Devices (IEDs),

NITRATES, NITRITES, NITROAMINES, and NITROESTERS—In examples, and without limitation, the colorimetric reagent can be an aromatic amine that is configured to detect nitrates, nitrites, nitroamines, or nitroesters. The colorimetric reagent enables and enhances the diazotization of nitrites, nitrates, nitroamines and nitroesters, which may be present in pre- and post-blast gun powder(s), conventional and home-made explosives and pre-cursors to explosives and gun powders, which may or may not be used in the bullet constructions, weapons and Improvised Explosive Devices (IED's). In an embodiment, the aromatic amine can be selected from sulfanilamide, sulfanilic acid, 4-Nitroaniline, 3-Nitroaniline, 2-Nitroaniline, 2-Fluoro-5-nitroaniline, 3-Aminobenzenesulfonic acid, 2-Aminobenzenesulfonic acid, 2,4-Diaminobenzenesulfonic acid, 4-Aminobenzoic acid, 3-Aminobenzoic acid, 4-Aminophthalic acid, Methyl 4-aminobenzoate, 3-Fluoro-4-aminobenzoic acid, 2-Aminobenzoic acid, 2-Amino-3-fluorobenzoic acid, 2-Amino-4-fluorobenzoic acid, 2-Amino-5-fluorobenzoic acid, 2-Amino-6-fluorobenzoic acid, 2-Amino-4,5-difluorobenzoic acid, 2-Amino-3-methylbenzoic acid, 2-Amino-5-methylbenzoic acid, 2-Amino-6-methylbenzoic acid, or naphthols.

In further embodiments, a dry powder of the colorimetric reagent can be combined with a dry powder of an acidic or alkaline catalyst to form a dry powder reagent blend. The catalyst can enables and enhances the diazotization of nitrites, nitrates, nitroamines, and nitroesters which may be present in pre- and post-blast gun powder(s), conventional and home-made explosives and pre-cursors to explosives and gun powders, which may or may not be used in the bullet constructions, weapons and Improvised Explosive Devices (IED's). Without limitation, the catalyst may be selected from oxalic acid, sodium hydrogen sulfate, citric acid, toluene sulfonic acid, sulfamic acid, sulfanilic acid, 3-Aminobenzenesulfonic acid, 2-Aminobenzenesulfonic acid, 2,4-Diaminobenzenesulfonic acid, 4-Aminobenzoic acid, 3-Aminobenzoic acid, 4-Aminophthalic acid, 3-Fluoro-4-aminobenzoic acid, 2-Aminobenzoic acid, 2-Amino-3-fluorobenzoic acid, 2-Amino-4-fluorobenzoic acid, 2-Amino-5-fluorobenzoic acid, 2-Amino-6-fluorobenzoic acid, 2-Amino-4,5-difluorobenzoic acid, 2-Amino-3-methylbenzoic acid, 2-Amino-5-methylbenzoic acid, 2-Amino-6-methylbenzoic acid, benzoic acid, sodium tetra borate, cationic salts of bicarbonate, carbonate, sulphate, or the like.

In further embodiments, dry powders of the colorimetric reagent and the catalyst can be combined with a dry powder of an azo-coupling compound to form a dry powder reagent blend. The azo coupling compound enables and enhances generation of visual color development with the catalyst (e.g., a diazotization compound) and nitrite ion from nitrites, nitrates, nitroamines and nitroesters, which may be present in pre- and post-blast gun powder(s), conventional and home-made explosives and pre-cursors to explosives and gun powders, which may or may not be used in the bullet constructions, weapons and Improvised Explosive Devices (IEDs). In examples, and without limitation, it may be selected from N,N,dimethyl-1-naphthylamine, N-1-naphthyl-ethylene-diamine dihydrochloride, 8-amino-1-naphthol-5-sulphonic acid, and 1-dimethylamino naphthalene-5-sulphonic acid.

In additional embodiments, dry powders of the colorimetric reagent, the catalyst, and the azo-coupling compound can be combined with a dry powder of a reducing compound to form a dry powder reagent blend. The reducing compound enables and enhances generation of nitrite ion from nitrites, nitrates, nitroamines, and nitroesters, which may be present in pre- and post-blast gun powder(s), conventional and home-made explosives and pre-cursors to explosives and gun powders, which may or may not be used in the bullet constructions, weapons and Improvised Explosive Devices (IED's). Without limitation, the reducing compound may be selected from zinc powders, iron powders, copper powders, or aluminum powders.

OXIDIZERS—In an embodiment, colorimetric reagent can include a compound able to undergo redox bathochromic shift and/or redox colorimetric reaction, enabling and enhancing the reduction of oxidizing compounds which may be present in pre- and post-blast gun powder(s), conventional and home-made explosives and pre-cursors to explosives and gun powders, which may or may not be used in the bullet constructions, weapons and Improvised Explosive Devices (IEDs). Without limitation, colorimetric reagent may be selected from methylene blue, potassium iodide starch, N,N′-diethyl-p-phenylenediamine sulfate, N,N,dimethyl-1-naphthylamine, N-1-naphthyl-ethylene-diamine dihydrochloride, alpha-naphthol, sulfanilic acid, copper sulphate, iron sulphate, or ferricyanide/cyanate salts.

METALS—In an embodiment, the colorimetric reagent can include a compound able to undergo redox bathochromic shift, colorimetric chelation, and/or colorimetric reaction with heavy metal cations, enabling and enhancing the color detection of metal compounds which may be present in pre- and post-blast gun powder(s), conventional and home-made explosives and pre-cursors to explosives and gun powders, which may or may not be used in the bullet constructions, weapons and Improvised Explosive Devices (IED's). Without limitation, the colorimetric reagent may be selected from Sodium Rhodizonate, rubeanic acid, dithizone, alizarin red s, salicylaldoxime, dithiooxamide, 2,9-dimethyl-4,7-diphenyl-1,10-phenanthroline, zinc dibenzyldithiocarbamate (ZDBT). ZDBT, oxalydihydrazide-[bis(2-hydroxyethyl) dithiocarbamate, Neocuproine, Bathocuproine, nickel diethyldithio phosphate, Oxalyldihydrazide, Trimethyl arsonium iodide, 2-Carboxy-2′-hydroxy-5′-sulfoformazylbenzene and its salts, or Chlorindazone DS.

In other embodiments, the solvent(s) should enable the dissolution of all components (e.g., dry chemical reagents (a)-(q) and GSR or ER (i)-(vii) above), thus facilitating interaction of all species and a chemical reaction producing a colorimetric indication for GSR or ER.

In embodiments, the solvent(s) may be selected from but, are not limited to, aqueous and non-aqueous solvents. In examples, the solvents, may be selected from, but are not limited to, water, dimethyl sulfoxide, or alcohols.

NITROAROMATICS—In embodiments, the kits and methods described herein provide for blending and homogenising dry powder reagents together to form a dry powder reagent blend. The dry powder reagent blend can be configured to detect nitroaromatics present in GSR and ER. In examples, without limitation, the dry powder reagent blend may be prepared in the ratio of (a) alkaline and nucleophilic reagent(s) sodium silicate (1 part) or sodium teraborate (1 part) or hydroxide salts (1 part) or N,N,dimethyl-1-naphthylamine (1 part) or N-1-naphthyl-ethylene-diamine dihydrochloride (1 part) or sodium borohydride (1 part) admixed with (5 parts) of any dry powdered excipient which allows dilution for packaging. Examples of the excipients can include, without limitation, metal carbonates, fumed silica, starches and the like. The process of mixing the dry powders can be achieved by placing the correct mass of each dry powder into any ball mill and with gentle agitation for some period of time, mixing and crushing the reagents until a homogenized dry mix is achieved.

NITRATES, NITROAMINES, NITRITES, and NITROESTERS—In embodiments, the kits and methods described herein provide for blending and homogenising the dry powder reagents together to form a dry powder reagent blend. The dry powder reagent blend can be configured to detect nitrates, nitroamines, nitrites, and nitroesters present in GSR and ER. In examples, without limitation, the dry powder reagent blend may be prepared in the ratio of (a) aromatic amine (1 parts), (b) an acidic catalyst (5 parts), (c) azo coupling compound (1 part) and (d) a reducing compound (1 part). The process of mixing the dry powders can be achieved by placing the correct mass of each dry powder into any ball mill and with gentle agitation for some period of time, mixing and crushing the reagents until a homogenized dry mix is achieved (e.g. consistent light grey coloration).

OXIDIZERS—In embodiments, the kits and methods described herein provide for blending and homogenising the dry powder reagents together to form a dry powder reagent blend. The dry powder reagent blend can be configured to detect oxidizers present in GSR and ER. In examples, without limitation, the dry powder reagent blend may be prepared in the ratio of colorimetric redox/chelation reagents methylene blue (1 part) or potassium iodide starch (1 part) or N,N′-diethyl-p-phenylenediamine sulfate (1 part) or N,N,dimethyl-1-naphthylamine (1 part) or N-1-naphthyl-ethylene-diamine dihydrochloride (1 part) or alpha-naphthol (1 part) or sulfanilic acid (1 part) or copper sulphate (1 part) or iron sulphate (1 part) or ferricyanide/cyanate salts (1 part) admixed with (5 parts) of any dry powdered excipient which allows dilution for packaging, for example and without limitation fumed silica, starches and the like. The process of mixing the dry powders can be achieved by placing the correct mass of each dry powder into any ball mill and with gentle agitation for some period of time, mixing and crushing the reagents until a homogenized dry mix is achieved.

METALS—In embodiments, the kits and methods described herein provide for blending and homogenising the dry powder reagents together to form a dry powder reagent blend. The dry powder reagent blend can be configured to detect metals present in GSR and ER. In examples, without limitation, the dry powder reagent blend may be prepared in the ratio of any one of the following as (1 Part) Sodium Rhodizonate, rubeanic acid, dithizone, alizarin red s, salicylaldoxime, dithiooxamide, 2,9-dimethyl-4,7-diphenyl-1,10-phenanthroline, zinc dibenzyldithiocarbamate (ZDBT). ZDBT, oxalydihydrazide-[bis(2-hydroxyethyl) dithiocarbamate, Neocuproine, Bathocuproine, nickel diethyldithio phosphate, Oxalyldihydrazide, Trimethyl arsonium iodide, 2-Carboxy-2′-hydroxy-5′-sulfoformazylbenzene and its salts, Chlorindazone DS, to (5 parts) of any dry powdered excipient which allows dilution for packaging, for example and without limitation fumed silica, starches and the like. The process of mixing the dry powders can be achieved by placing the correct mass of each dry powder into any ball mill and with gentle agitation for some period of time, mixing and crushing the reagents until a homogenized dry mix is achieved.

In embodiments, the dry powder blend may be packaged in a paper or synthetic form fill seal sachet, or breakable ampoule, or blister pack, or vials, bottles, tubes, all of which provide storage, downstream packaging, shipping, until the powder is required for use.

In embodiments, the kits and methods described herein provide for the dry powder blend to be formed into articles by pressure die or extrusion molding of a mixture of the dry powder blend including inert binders and/or release agents. By way of example and without limitation, one method of this process may involve the dry powder blend being mixed with, for example, one or more of microcrystalline wax, cellulose, poly-vinyl alcohol, polyvinylpyrrolidone, starch, SDS, and the like. The mix can be poured into a pill press to form small wafer thin dry reagent discs, ready for use in detection of explosive residues which may be present in pre- and post-blast gun powder(s), conventional and home-made explosives and pre-cursors to explosives and gun powders, which may or may not be used in the bullet constructions, weapons and Improvised Explosive Devices (IEDs). Alternatively, the mix may be rolled into sheets, or extruded into pellets. This may be achieved with convention pressure forming machinery, well known in the art.

In embodiments, the pressed formed reagent article, would ordinarily be package in a suitable format for safe storage, shipping and later use, for example form fill sealed in hermetically sealed light, air and moisture proof packaging.

In embodiments, the kits and methods described herein provide for the dry homogenized powder being dry impregnated into a webbed matrix of natural or synthetic fibers carrier. By way of example and without limitation, one method of this process may involve the homogenized dry powder, being mixed with a fibrous material (e.g., cotton swab or wipe). The micronized dry powder particles are held and affixed within the fiber matrix. Alternatively, the mix may be rolled and embedded into the fiber matrix of sheets. This may be achieved with conventional pressure, rolling and fiber article forming machinery, well known in the art.

In embodiments, the kits and methods described herein provide for the dry homogenized powder(s) described in previous sections being added to a solvent(s), causing gelling of the liquefied powder(s) suspension for printing onto a solid support. The solid support may be any surface, which allows for printing to the surface and subsequent drying of the surface to remove said solvent. The printing method is any mass manufacture industrial printing methodology known in the art, including and without limitation flexographic, stamping, screen printing systems.

In embodiments, the kits and methods described herein provide for the dry homogenized powder(s) described in previous sections being added to and dissolved in a suitable solvent which creates a homogenous liquid, which is suitable for bibulous carrier impregnation, known in the art as a dip-n-dry process. In this case the bibulous carrier can be any matrix which absorbs the dissolved solution and allows evaporation of the solvent leaving behind a dry impregnated carrier/reagent matrix.

In embodiments, the kits and methods described herein provide for suitable packaging for long term shipping and storage prior to end use for any of the above disclosed modalities of presenting the kits. For example and without limitation, form fill seal polyethylene (PE), polyethylene terephthalate (e.g., Mylar®), bi-axially oriented polypropylene (BOPP), or polyvinyl chloride (PVC) pouch materials.

In embodiments, the kits and methods described herein provide for a solvent(s) which facilitate dissolution and reaction of all reagents and residues, which may give rise to a colorimetric indication. The solvent(s) selected is provided in a pre-wetted absorbent material (e.g., a swab, wipe, or towelette) or in a snap swab or pop swab modality.

In embodiments, the pre-wetted solvent swab would be produced by simple dipping a dry cotton swab into a solution of the solvent(s) and then inserted into a package in a suitable format for safe storage, shipping and later use, for example form fill sealed in hermetically sealed light, air and moisture proof packaging.

In embodiments, the methods described herein provide that, once the suspect residue or surface to be tested, has been located, both the dry powder reagent packaging and pre-wetted swab packaging are opened. The pre-wetted swab is removed from the shipping packaging, with the operator holding the shaft of the swab, it is firmly rubbed across and into said residue or surface, for approximately 10 seconds. This is achieved without need for operator to touch suspect residue or surface.

In embodiments, the sampled swab is transferred to the opened homogenized dry powder packaging and inserted into the packaging to allow the wet sampled swab to impact and mix with the homogenized dry powder reagent. Agitation may be provided by the operator, to facilitate mixing and chemical reaction. This is all achieved without need for the operator to come into contact with dry powder reagents(s), solvent(s) and suspect residue(s).

In embodiments, the method provides that the operator views both the swab tip and reaction mix for the following indications: (A) Positive presumptive colorimetric indication for the presence of GSR is rapid formation of obvious purple-pink-red color formation, (B) Negative result—no color change. Positive color indications for GSR should occur and be visible, within 5 to 10 seconds.

In an embodiment, a portable detection kit for identifying the presence of gunshot residue (GR) and explosive residue (ER) is provided. The kit can include a colorimetric reagent(s), a solvent(s), and a swab(s). The colorimetric reagent(s) and solvents(s) and a swab(s) can be provided in hermetically sealed light, air and moisture proof packaging.

In another embodiment, the GSR and ER can include any discharge from weapons and explosives.

In another embodiment, the GSR or ER can be liquids, gels or solid residues that are pure or admixed with clandestine cutting or diluting agents.

In another embodiment, the colorimetric reagent(s) in combination with the solvent(s) are configured to undergo physico-chemical interaction with GSR and ER, producing colored reaction product(s), resulting in a visible color change.

In another embodiment, the GSR or ER is Metal Particulate (MP) and the swab is a pre-wetted cotton swab for the collection of MP.

In another embodiment, the swab is pre-wetted with a solution comprising the solvent(s) and the colorimetric reagent(s). The solution can be a volume of 0.05 to 0.2 mL of a saturated sodium tetraborate/water solution and the pre-wetted swab can be individually packaged into a hermetically sealed sachet.

In another embodiment, the colorimetric reagent(s) can be selected from the group consisting of: Sodium Rhodizonate, rubeanic acid, dithizone, alizarin red s, salicylaldoxime, dithiooxamide, 2,9-dimethyl-4,7-diphenyl-1,10-phenanthroline, zinc dibenzyldithiocarbamate (ZDBT). ZDBT, oxalydihydrazide-[bis(2-hydroxyethyl) dithiocarbamate, Neocuproine, Bathocuproine, nickel diethyldithio phosphate, Oxalyldihydrazide, Trimethyl arsonium iodide, 2-Carboxy-2′-hydroxy-5′-sulfoformazylbenzene and its salts, Chlorindazone DS.

In another embodiment, the colorimetric reagent(s) can be selected from the group consisting of 2-Carboxy-2′-hydroxy-5′-sulfoformazylbenzene and its salts.

In another embodiment, the kit can further include a bibulous carrier including a solution of the colorimetric reagent(s) and the solvent(s) absorbed therein. The solution can include 0.2 grams of 2-Carboxy-2′-hydroxy-5′-sulfoformazylbenzene or its salts in 70 mL of ethanol. The bibulous carrier can be chromatography filter paper.

In another embodiment, the GSR or ER is a nitroaromatic (NA) and the swab can be pre-wetted for the collection of NA. As an example, the swab can be pre-wetted with a solution including the solvent and the colorimetric reagent(s). The solution can be a volume (e.g., about 0.05 to about 0.2 mL) of a 75% dimethylsulfoxide (DMSO) and 25% isopropyl alcohol solution and the pre-wetted swab can be individually packaged into a hermetically sealed sachet.

In another embodiment, the colorimetric reagent(s) can be selected from the group consisting of sodium silicate, sodium teraborate, hydroxide salts, N,N,dimethyl-1-naphthylamine, N-1-naphthyl-ethylene-diamine dihydrochloride, and sodium borohydride.

In another embodiment, the kit can further include comprising a solid support substrate including a solution of the colorimetric reagent(s) and the solvent(s) applied thereon. In an example, the solution can include 1 gram of sodium tetraborate in 100 mL sodium silicate. The solid support substrate can be 300 gsm card or synthetic paper.

In another embodiment, the GSR or ER can be at least one of Nitrates, Nitrites, Nitroamines Nitroesters (NNNN) and the swab can be a pre-wetted cotton swab for the collection of NNNN. As an example, the swab can be pre-wetted with the solvent(s). The solvent(s) can be a volume (e.g., about 0.05 to about 0.2 mL) of a 75% DMSO and 25% isopropyl alcohol and the pre-wetted swab can be individually packaged into a hermetically sealed sachet.

In another embodiment, the colorimetric reagent(s) is selected from the group consisting of: sulfanilamide, sulfanilic acid, 4-Nitroaniline, 3-Nitroaniline, 2-Nitroaniline, 2-Fluoro-5-nitroaniline, 3-Aminobenzenesulfonic acid, 2-Aminobenzenesulfonic acid, 2,4-Diaminobenzenesulfonic acid, 4-Aminobenzoic acid, 3-Aminobenzoic acid, 4-Aminophthalic acid, Methyl 4-aminobenzoate, 3-Fluoro-4-aminobenzoic acid, 2-Aminobenzoic acid, 2-Amino-3-fluorobenzoic acid, 2-Amino-4-fluorobenzoic acid, 2-Amino-5-fluorobenzoic acid, 2-Amino-6-fluorobenzoic acid, 2-Amino-4,5-difluorobenzoic acid, 2-Amino-3-methylbenzoic acid, 2-Amino-5-methylbenzoic acid, 2-Amino-6-methylbenzoic acid, naphthols, oxalic acid, sodium hydrogen sulfate, citric acid, toluene sulfonic acid, sulfamic acid, sulfanilic acid, 3-Aminobenzenesulfonic acid, 2-Aminobenzenesulfonic acid, 2,4-Diaminobenzenesulfonic acid, 4-Aminobenzoic acid, 3-Aminobenzoic acid, 4-Aminophthalic acid, 3-Fluoro-4-aminobenzoic acid, 2-Aminobenzoic acid, 2-Amino-3-fluorobenzoic acid, 2-Amino-4-fluorobenzoic acid, 2-Amino-5-fluorobenzoic acid, 2-Amino-6-fluorobenzoic acid, 2-Amino-4,5-difluorobenzoic acid, 2-Amino-3-methylbenzoic acid, 2-Amino-5-methylbenzoic acid, 2-Amino-6-methylbenzoic acid, benzoic acid, sodium tetra borate, cationic salts of bicarbonate, carbonate, sulphate and the like, N,N,dimethyl-1-naphthylamine, N-1-naphthyl-ethylene-diamine dihydrochloride, 8-amino-1-naphthol-5-sulphonic acid, and 1-dimethylamino naphthalene-5-sulphonic acid, zinc powder, iron powder, copper powder, and aluminum powder.

In another embodiment, wherein the colorimetric reagent(s) is provided a solution with the solvent(s), where the solution includes (i) 1 gram of sulfanilamide, (ii) 1 gram of sulfanilic acid, (iii) 1 gram of N-1-naphthyl-ethylene-diamine dihydrochloride, (iv) 50 grams of citric acid in 500 mL of aqueous alcohol solvent. The aqueous alcoholic solvent can include water and isopropyl alcohol in a predetermined ratio. In another embodiment, the kit can also include a solid support substrate including the solution applied thereon. The solid support substrate can be 300 gsm card or synthetic paper and packaged into a hermetically sealed sachet.

In another embodiment, the GSR or ER is an oxidizer (OX) and the swab is a pre-wetted cotton swab for the collection of OX. The swab can be pre-wetted with a solution including the solvent(s) and the colorimetric reagent(s). The solvent(s) can be a volume (e.g., about 0.05 mL to about 0.2 mL) of water.

In another embodiment, the colorimetric reagent(s) can be selected from the group consisting of: methylene blue, potassium iodide starch, N,N′-diethyl-p-phenylenediamine sulfate, N,N,dimethyl-1-naphthylamine, N-1-naphthyl-ethylene-diamine dihydrochloride, alpha-naphthol, sulfanilic acid, copper sulphate, iron sulphate, and ferricyanide/cyanate salts.

In another embodiment, the colorimetric reagent(s) is methylene blue.

In another embodiment, the kit can further include a solid support substrate including a solution of the colorimetric reagent(s) and solvent(s) applied thereon. The solution can include 0.2 gram of methylene blue in 100 mL of ethanol solvent. In an embodiment, the solid support substrate can be 300 gsm card or synthetic paper and the solid support substrate can be packaged into a hermetically sealed sachet.

In another embodiment, the solvent(s) can be configured to dissolve the colorimetric reagent(s), the GSR, and ER.

In another embodiment, the solvent(s) include aqueous and non-aqueous solvents. The solvent(s) can include at least one of water, dimethyl sulfoxide, or alcohols.

In another embodiment, the swab(s) can be a cotton swab that is pre-wetted with a predetermined amount (e.g., about 0.1 mL to about 0.5 mL) of the solvent(s). The pre-wetted swab can be individually packaged in a hermetically sealed sachet.

In another embodiment, the colorimetric reagent(s) include a mixture of at least two dry powder reagents. The mixed dry powder reagents can exhibit a homogenized micronized size within the range from about 0.1 micron to about 30 micron.

In another embodiment, the kit can include a sealed sachet containing about 0.1 grams to about 1 grams of the colorimetric reagent(s).

In another embodiment, the kit can include a solid support substrate including a solution of the colorimetric reagent(s) applied thereon.

In another embodiment, the kit can include a sealed sachet containing the colorimetric reagent(s), the solvent(s), and the swab(s). The sachet can be formed from paper, PET12 um, AL7 um, or PE50.

In another embodiment, a method of detecting gunshot residue (GSR) or explosive residue (ER) is provided the method can include providing a portable detection kit. The kit can include a colorimetric reagent(s), a solvent(s), and a swab(s). The colorimetric reagent(s) and solvents(s) and a swab(s) can be provided in hermetically sealed light, air and moisture proof packaging. The kit can further include hermetically sealed sachets containing the swab pre-wetted with a water solvent and dry powder of the colorimetric reagent(s). The method can further include locating a residue or surface suspected to include GSR and/or ER. The method can also include opening both the sealed sachets containing the colorimetric reagent(s) and the pre-wetted swab.

In another embodiment, the method can further include removing the pre-wetted swab from its sachet, applying a swab tip to the suspect residue and/or surface, and rubbing the swab tip on the residue and/or surface to thereby dissolve and collect GSR and/or ER, yielding a sampled swab.

In another embodiment, the method can further include contacting the sampled swab with the colorimetric reagent(s) for a period of at least 10 seconds.

In another embodiment, the method can additionally include monitoring both the swab tip and colorimetric reagent(s) for development of a presumptive colorimetric indication for the presence of GSR and/or ER. The presumptive indications can include: (metal negative—purple to white)(metals positive—purple to blue), (nitroaromatics (NA) negative—white to white)(nitroamine NA positive—white to pink/red), (Nitrates, Nitrites, Nitroamines or Nitroesters (NNNN) negative—white to white)(NNNN positive—white to pink/red), (oxidizers (OX) negative—blue to blue or white)(OX positive—blue to pink/purple)

In an embodiment, a method of detecting gunshot residue (GSR) or explosive residue (ER) is provided. The method can include providing a portable detection kit. The kit can include a colorimetric reagent(s), a solvent(s), and a swab(s). The colorimetric reagent(s) and solvents(s) and a swab(s) can be provided in hermetically sealed light, air and moisture proof packaging.

The swab(s) can include a hollow shaft and a tip containing a fiber matrix and the colorimetric reagent(s) can include dry powder reagent(s) embedded into the tip fiber matrix. A water solvent can be contained within the hollow shaft. The kit can further include the swab(s) and the colorimetric reagent(s) hermetically sealed within respective portions of a sachet.

In another embodiment, the method can further include locating a residue or surface suspected to include GSR and/or ER, and opening the hermetically sealed sachet containing the swab(s).

In another embodiment, the method can also include removing the swab(s) from its sachet, breaking the hollow shaft of the swab to releasing the water solvent into the tip of the swab, applying the swab tip to the suspect residue and/or surface; and rubbing the swab tip on the residue and/or surface (e.g., for at least about 10 seconds) to dissolve and collect GSR and/or ER, yielding a sampled swab.

In another embodiment, the method can further include monitoring both the swab tip and the rubbed surface to detect an indication comprising one of: (A) a purple-pink-red color formation representing positive presumptive colorimetric indication for the presence of GSR and/or ER or (B) a white swab tip/no color change representing a negative result.

In an embodiment, a method of detecting gunshot residue (GSR) or explosive residue (ER) is provided. The method can include providing a portable detection kit. The kit can include a colorimetric reagent(s), a solvent(s), and a swab(s). The colorimetric reagent(s) and solvents(s) and a swab(s) can be provided in hermetically sealed light, air and moisture proof packaging. The swab(s) can include a swab that is pre-wetted with a water solvent and is sealed in a sachet. The colorimetric reagent(s) can include one of dry powder reagents sealed in a sachet, a sheet sealed in a sachet, dry powder reagent(s) sealed in a breakable ampule or blister pack, a tablet or pellet sealed in a sachet. The method can further include locating a residue or surface suspected to include GSR and/or ER, and opening the sachet containing the pre-wetted swab and the sachet, ampule, or blister pack containing the colorimetric reagent(s).

In another embodiment, the method can further include removing the pre-wetted water swab from its sachet, applying a swab tip to the suspect residue and/or surface, and rubbing the swab tip on the residue and/or surface (e.g., for at least about 10 seconds) to thereby dissolve and collect the GSR and/or ER, yielding a sampled swab.

In another embodiment, the method can further include inserting the sampled swab into the opened sachet, blister pack, or ampoule, contacting the sampled swab with the powder, sheet, tablet or pellet containing the colorimetric reagent(s) (e.g., for a period of at least about 10 seconds), and waiting for the development of a positive colorimetric reaction.

In another embodiment, the method can further include observation of both the swab tip and the colorimetric reagent(s) to detect an indication comprising one of: (A) a purple-pink-red color formation representing positive presumptive colorimetric indication for the presence of GSR and/or ER or (B) a white swab tip/no color change representing a negative result.

Other aspects of the embodiments of the disclosure are discussed infra.

### BRIEF DESCRIPTION OF THE DRAWINGS

Other objectives of the disclosed embodiments will appear in the description and claims, with reference being made to the accompanying drawings, which form part of the specification.

FIG. 1 illustrates a swab made in accordance with embodiments of the disclosure. The swab can include a handle 100 formed from a plain white card or plastic (e.g., polypropylene) and a tip 102 formed from cotton or synthetic fiber. The tip 102 can include (i) homogenized dry powder reagents 104 embedded within its matrix, or it can be (ii) pre-wetted with a solvent(s).

FIG. 2 illustrates a fiber tipped snap swab made in accordance with embodiments of the disclosure. The swab includes a hollow plastic shaft 202 that is filled with any combination of solvent(s) 210. A portion of the shaft 202 is etched 200 and configured to snap 204 when twisted, to release the solvent 210 contained in hollow shaft 202 into a swab tip 206. The swab tip 206 includes dry homogenized dry powder reagents 104 embedded within its matrix.

FIG. 3 illustrates a fiber tipped pop swab made in accordance with embodiments of the disclosure; 300 is a flexible plastic hollow shaft pre-filled with any combination of solvent(s) 210. At location 302 the shaft 300 is squeezed and “popped”, releasing solvent(s) 210. A swab tip 304 includes dry homogenized dry powder reagents 104 embedded within its matrix.

FIG. 4 illustrates a sachet 400 made in accordance with embodiments of the disclosure, pre-filled with any combination of homogenized dry powder reagents 104.

FIG. 5 illustrates a blister pack 500 made in accordance with embodiments of the disclosure, pre-filled with any combination of combination of homogenized dry powder reagents 104.

FIGS. 6A-6B illustrate a breakable ampoule made in accordance with embodiments of the disclosure, pre-filled with homogenized dry powder reagents. FIG. 6A illustrates a plastic or glass or flexible polymer ampoule 600 pre-filled with said dry powder reagents (FIG. 6A). FIG. 6B illustrates a broken/snapped/squashed ampule 602 that releases homogenized dry powder reagent formulation 104.

FIGS. 7 and 8 illustrate pressed and formed articles made in accordance with embodiments of the disclosure. Through pressure and extrusion processes, the homogenized dry powder reagents in combination with binders and release agents can be encapsulated and formed into sheets 700 (FIG. 7), tablets or pellets 800 (FIG. 8).

FIG. 9 illustrates one exemplary embodiment of a kit format and packaging format made in accordance with embodiments of the disclosure. The homogenized dry powder reagent 104 is powder filled into a suitable first sachet 900. A pre-wetted swab 902 is inserted into suitable a second sachet 900′ and hermetically sealed. The sealed second sachet 900′ containing the pre-wetted swab 902 is placed inside the first sachet 900 which is subsequently hermetically sealed for shipping.

FIG. 10(a) illustrates another exemplary embodiment of a kit manufacturing format made in accordance with embodiments of the disclosure that of “dip-n-dry” pre-impregnated reagent bibulous carrier. A chromatography paper 1000 (e.g. Whatman #1 CP), is partially dipped into a reagent solution 1002 held in a dipping tray 1004. The reagent solution 1002 wicks and absorbs into the chromatography paper 1000. The wetted chromatography paper 100 is removed from the tray 1004 for subsequent drying by hot air stream.

FIG. 10(b) illustrates a chromatography paper 1000 including a portion that is impregnated with dried reagent 1002.

FIG. 10(c) illustrates the structure of FIG. 10(b) after guillotining into a desired end use shape (e.g., a strip).

FIG. 11 illustrates an exemplary embodiment of kit manufacturing format according to embodiments of the disclosure in the form of a printed dry reagent test strip 1100. The dry reagent powder mix 104 is added to and mixed with a solvent and suitable gelling agent, to produce a homogenous suspension and printed to any solid support substrate 1102 (e.g. 300 gsm card or Synthetic paper) which can facilitate large scale print manufacturing, using standard off the shelf equipment known in the art (e.g., flexographic or offset screen printing). The dried article (e.g. test strip 1100) may have one or many reagent dye test zones 1104 to detect and presumptively identify one or more unknown compounds.

### DETAILED DESCRIPTION

Embodiments of the present disclosure provide for improved systems and methods detection of Gun Shot Residue (GSR) and/or conventional and home-made explosive residue(s) (ER) and their pre-cursors.

### GSR and ER Detection

Contrary to current approaches for the presumptive identification of both GSR and ER, embodiments of the present disclosure provide, for the first time, that selected dry chemical reagents (e.g., (a) dyes (e.g. azo, formazan, mordant), (b) cyclic enols, (c) carbamates, (d) sulphonic acids, (e) imidazolines, (f) thio compounds, (g) diazotising reagents, (h) oximes, (i) phenylamines, (j) phenanthrolines, (k) hydrazides, (l) arsonium, (m) boron based compounds, (n) alkaline silicates, (o) hydroxide salts, (p) nucleophiles, or (q) metal salts) and solvents, can be successfully mixed and packaged in various combinations to provide a selective, cheap, mass producible, portable detection device for the presumptive identification of GSR and ER, and provide operator safety when using said device. Other advantages of embodiments of the disclosed systems and methods can prevent surface spoilage, damage and contamination, while preventing destruction of forensic evidence. The device disclosed produces colorimetric indications in seconds.

### Current Detection Methods

Commercially available colorimetric GSR detection kits are available. For example, Fisher Scientific®, Carolina®, Sirchie®, Safariland®, Innovating Science®, Tri-Tech Forensics, Edu-Lab®, Sandia National Laboratories, iDenta and many others, provide kits containing liquid reagent dropper bottles, powder reagents and alcohol swabs. Arrowhead Forensics, DetectaChem® and many others provide kits containing aerosolized liquid reagent spray cans or crushable glass ampoules filled with liquid reagents.

The aforementioned kits contain many un-desirable features. In one aspect, such kits can employ highly corrosive and hazardous liquids. In another aspect, such kits can employ glass bottles and ampoules which bulky, cumbersome and break, causing sharps inflicting cuts, exposing skin to direct hazardous chemical contact. In a further aspect, such kits can employ aerosolized spray cans, which create chemical fume and spray drift of hazardous chemicals. In an additional aspect, such kits can employ bulky packaging and large waste stream, with limited recycle potential. In a further aspect, these kits are difficult to air-freight, which adds significant cost to end users.

The aforementioned kits, during use, will leave residues of chemical reagents on surfaces and residues of forensic significance, thus contaminating crime scenes and destroying vital evidence and even damage surfaces, including but not limited to destruction of paint, keyboards and skin.

The aforementioned kits are excessively costly and kits generally have slow chemical reactivity, some requiring several minutes for a single analysis.

The field deployable kits highlighted above also suffer from a variety of manufacture and end use problems, including but not limited to:
(i) Construction of existing kits requires liquid dropper bottles, breakable glass or plastic ampoules, blister packs and pressurized aerosol spray cans filled with hazardous liquid reagents.
(ii) The presence of hazardous liquid reagents employed by existing kits and devices poses problems with manufacturing and exposure limitations, storage and handling, strict packaging requirements and significant shipping restrictions.
(iii) The volume or quantity of liquid reagent consumed during one single test performed by existing kits and devices is excessive and wasteful adding to costly, bulky and often overly complicated device construction design and packaging.
(iv) During use of existing kits and devices, operators may be exposed to sharps and hazardous liquid reagent splash or overspray.
(v) Existing kits and devices require multi step operations in order to complete a single test.
(vi) Existing kits and devices do not achieve the bench mark of true low cost mass manufacturing, which would be considered in the range of tens of millions of individual units per annum, with a commercial shelf life span of 3 to 5 years,

Without limitation, embodiments of the kits described herein overcome these highlighted limitations and achieve the necessary benchmarks.

### GSR and ER Analysis

Many methods for the identification of GSR and ER currently exist, including but not limited, to technical theory embodied in patent and scientific literature, laboratory procedures requiring highly sophisticated electronic equipment, and classical laboratory procedures all of which have limited or no field application, by way of example some of these are described below.

The first method for determining whether or not someone had fired a gun was the GSR paraffin test, also known as the dermal nitrate or diphenylamine test. Teodoro Gonzalez of the Criminal Identification laboratory in Mexico City first applied this test in the United States in 1933.[2] An excellent review of GSR analysis and detection techniques is given by Sebastian.[3] The above historical practise can be easily extended to detection and identification of person(s) having ER on their bodies and clothing.

Two common presumptive color change test are used for GSR and ER analysis. (1) The Griess reagent, which detects the Nitrite anion and (2) the Rhodozinate test, which identifies Lead based species.

GSR and ER is produced when the firearm and/or explosive is discharged and/or when a person holds a dirty weapon, bullets, casings, explosives, IED's, and/or pre- and post-blast materials and pre-cursors. The explosive discharge generates hot gasses as a result of gunpowder burning. The expelled gas and particles have little mass and begin to settle on surfaces in, on and around the immediate vicinity to the point of discharged.

It is well documented, that GSR and ER settles on the clothing and hands of the person firing the weapon or handling the object or materials containing or coated in the GSR and ER. Also, depending on distance the object or surface is from the point of discharge, GSR and ER may also settle on the object surface(s). Generally speaking, for the purposes of the current device and its limits of use and detection, it is unlikely that GSR and ER will travel further than 1.5 m in any direction, from the point of discharge.

### Current Techniques and Shortcomings

U.S. Pat. No. 6,613,576 describes methods for GSR detection from the surface of a firearm, using a pre-wetted towel or swab to scrape away the deposited reside from the surface. The swab is then placed into a plastic injection molded cassette device which contains sealed crushable, cylinders or ampoules, which contain liquid reagents including but not limited to sulfuric acid and solvent dissolved diphenylamine. The described technique has very in-efficient residue collection technique and has been developed for identification of lead, and not the Nitrite ion as disclosed in the current application. The ampoules are likely to break during transport and storage. Liquid reagents are hazardous and carcinogenic.

BR 102014004252 describes methods for detecting lead residues, discharged from a fired weapon and requires the use of one or more of hydrogen peroxide, hydrochloric and or sulphuric acids. It does not describe detection of the Nitrite anion as described in the current application and the use of hazardous liquid reagents is highly undesirable.

US 2017/0153180 describes methods for detecting a method for detecting lead species in GSR, by way of fluorescence spectroscopy. This methodology requires sophisticated electronic instrumentation and as such, is not applicable to colorimetric field tests, as described in the current application.

U.S. Pat. No. 6,613,576 describes methods for detecting lead species in GSR with diphenylamine and sulphuric acid reagents. The reagents are contained in chambers, which need to be physically broken or breached in order for the colorimetric reaction with the sample residue to occur. This methodology employs hazardous and carcinogenic liquid reagents, produces large amounts of waste and likely sharps, which can cut operators.

U.S. Pat. No. 7,410,612 describes methods for detecting explosive residues including GSR. The method requires sample to be physically removed or scooped from surfaces and placed inside a thick plastic or flexible chamber which contains breakable ampoules of liquid reagents including sulphuric acid. The method described uses hazardous liquid reagents, and ampoules likely made of glass, both of which can cause serious harm and injury to operators.

U.S. Pat. No. 7,499,808 describes methods for detecting nano-particles of GSR on surfaces utilising sophisticated instrumentation including scanning electron microscopy and as such, is not applicable to colorimetric field tests, as described in the current application.

U.S. Pat. No. 9,880,092 describes methods for detecting GSR utilising a plastic injection molded rotatable housing which has one or many crushable ampoules welded into the body of the rotatable housing. This device utilises many liquid reagents, most of which are hazardous. It presents many manufacturing, shipping, storage and use complications and disposal hazards.

U.S. Pat. No. 10,260,999 describes methods for the collection of particulate residues, utilising specialised vacuum suction devices, which may be used for GSR residue sample. The disclosure is not for the detection and identification of GSR.

WO 2010/105326 describes methods for detecting the expelled residues of pre-fabricated luminescent gunpowder and lead based projectiles. This methodology requires sophisticated electronic instrumentation and as such, is not applicable to colorimetric field tests, as described in the current application.

U.S. Pat. No. 3,748,096 describes a complicated multi-step method to produce a dry pre-impregnated bibulous carrier for the identification of copper ions in environmental samples. The formulation described requires many individual chemical reagent including (i) the complexing agent, (ii) buffering agent(s) (iii) surfactant(s), and (iv) reducing agents. The colorimetric or complexing agent include 2,2-diquinoline; 2,9-dimethyl-1,10-phenanthroline; 2,9-dimethyl-4,7-diphenyl-1,10-phenanthroline; the disodium salt of 2,9-dimethyl-4,7-bis[S-phenylsulfonic acid]-1,10-phenanthroline, none of which are utilised in the disclosed embodiments, and the method also requires the use of highly undesirable hazardous organic solvents including benzene and toluene. This device is also not suitable or capable of use in GSR and ER detection. The method described in embodiments of the disclosure is a single step manufacture process, utilising a single chemical reagent, and removes the need for any hazardous organic solvent.

U.S. Pat. No. 5,620,658 describes yet another device designed specifically for copper and iron analysis in environmental samples. Described is a dry reagent impregnated bibulous carrier, which requires multi step, multi reagent and multi component system for the production of a portable copper detector. The method describes the need for (i) complexing agent: 5-(4-dimethylaminobenzylidene)rhodamine, and useful for analysis of iron ions include 2,4,6-tri(2-pyridinyl)-1,3,5-triazine (TPTZ). (ii) Reducing agents. (iii) Buffering reagents and (iv) surfactants. The reagents described are not utilised in embodiments of the disclosure, and the method also requires the use of highly undesirable hazardous organic solvents including benzene and toluene. This device is also not suitable or capable of use in GSR and ER detection. The method described in embodiments of the disclosure is a single step manufacture process, utilising a single chemical reagent, and removes the need for any hazardous organic solvent.

### Need in the Art and Failure of Others

As described herein, the current state of the art and identified patents and reference literature fail to identify a simple non-hazardous, rapid and reliable field deployable test for GSR and ER which provides a highly selective, cheap, mass producible, highly portable detection device for the presumptive identification of GSR and ER, enhancing operator safety when using the device, prevent surface spoilage, damage and contaminations, while preventing destruction of forensic evidence. Also many of the previous patents and reference literature requires long periods of time and additional equipment not described in order to facilitate the full function of the devices and techniques described. The device disclosed in embodiments of the disclosure does not require additional equipment and produces colorimetric indications in seconds.

The current state of the art techniques fail to provide operator safety.

The current state of the art techniques and literature fail to provide sampling methods, which do not damage surfaces and destroy significant forensic evidence.

The current state of the art techniques fail to provide simple single step manufacture techniques for the identification of individual classes of GSR and ER.

The current state of the art techniques generally require excessive use of multi reagent and organic solvent systems to facilitate manufacture of the device.

The preceding summary, of patent references and public domain literature and disclosures, does not intend to limit itself to these examples. They are provided only as a point of reference in the difference between laboratory equipment and procedures and true single step, cheap mass producible, field deployable presumptive colorimetric test kit for the detection of GSR which limits exposure of operators to the hazards as disclosed in the current application.

### Absorbent Materials

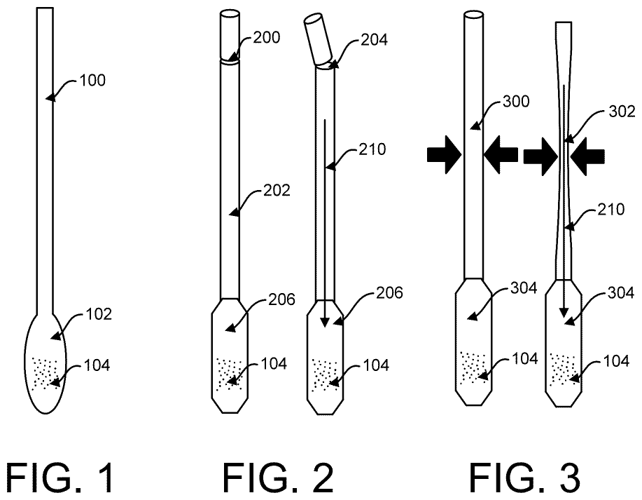

In accordance with embodiments of the disclosure and without limitation, in one embodiment, the absorbent material is a pre-wetted cotton swab, as illustrated in FIG. 1. The cotton swab includes a handle 100 and a tip or head 102 at one end of the handle 100. The pre-wetting can be achieved by simple dip and/or rapid immersion of the cotton swab tip into large volume pre-mixed co-solvent vat or micro-jet spray or similar. Pre-wetting can be a fully automated process utilizing conventional conveyor, hopper, spray machinery, which are known in the art.

In another embodiment and without limitation the absorbent material is a “snap cotton swab,” illustrated in FIG. 2. The swab includes a hollow swab handle 202 that is pre-filled in fully automated commercial fill processes with a solvent 210, as described in preceding sections of this disclosure. A swab tip 206 at one end of the swab handle 202 has an etched groove 200 that is applied during swab manufacture. The etched groove 200 is configured to be easily snapped 204 between thumb and forefinger, releasing said shaft contents down and into the swab tip (206). The swab tip 206, as described in the preceding sections of this disclosure, can have the homogenized dry reagent powders 104 lodged within the tip fiber matrix.

In yet another embodiment and without limitation the absorbent material is a “pop cotton swab,” as illustrated in FIG. 3. A hollow flexible swab handle 300 is pre-filled (e.g., in fully automated commercial fill processes) with the solvent 210, as described in preceding sections of this disclosure. The swab handle 300 is easily squeezed (e.g., at location 302) and small closure contained with hollow shaft handle 300 broken or “popped” between thumb and forefinger, releasing the solvent 210 down and into a swab tip 304. In alternative embodiments, the swab tip 304 of the pop cotton swab, as described in the preceding sections of this disclosure, can have the homogenized dry reagent powders 104 lodged within the tip fiber matrix.

In yet another embodiment and without limitation the absorbent material is a “wipe”. The “wipe” is made of any natural or synthetic polymeric fibers and pre-wetted with a solvent, as described in preceding sections of this disclosure. The “wipe” is simply applied to the suspect residues and pressed into or wiped across said residue for collection and subsequent presumptive identification of said residues, by mixing with the homogenized dry powder reagents.

In accordance with embodiments of the disclosure, and without limitation, a suitable way of providing the device is by pre-wetting a single head cotton swab, which contains, solvent, surfactant, dye.

### Free-Standing Dry Powder Colorimetric Reagent Blends

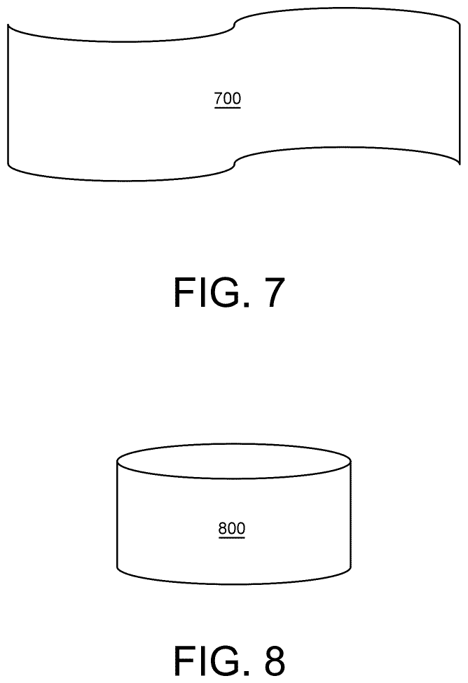

FIGS. 7 and 8 illustrate pressed and formed articles made in accordance with embodiments of the disclosure. Through pressure and extrusion processes, the homogenized dry powder reagents in combination with binders and release agents can be encapsulated and formed into sheets 700 or tablets/pellets 800.

### Containers

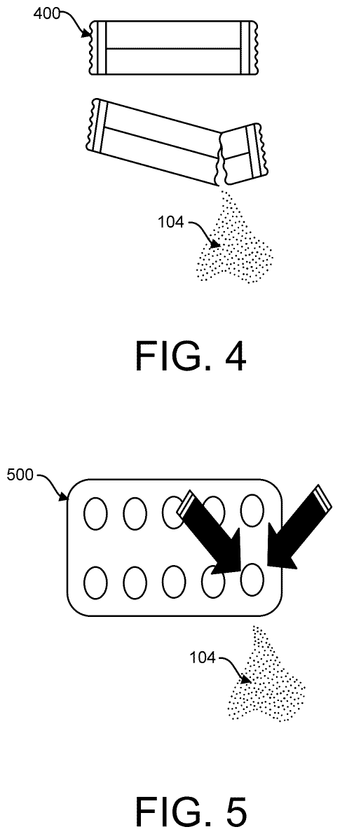

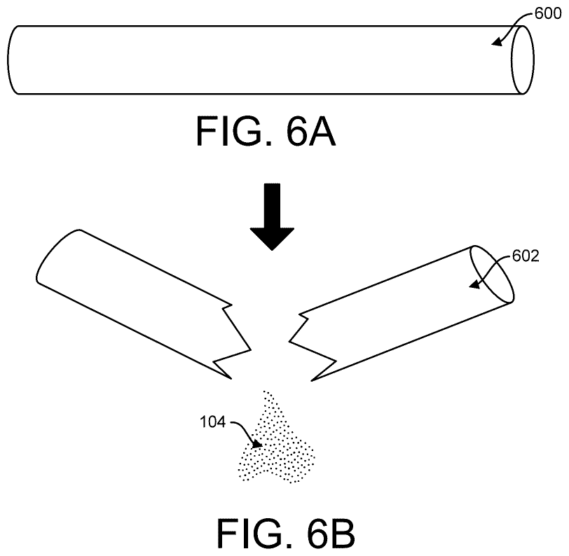

An ampoule 600 (FIG. 6), a blister pack 500 (FIG. 5), or suitable hermetically sealable sachet 400 can be used to separately contain a solvent and homogenized dry powder colorimetric reagent(s) 104, as described in preceding sections of this disclosure. A sachet 400 suitable for receipt of dry powder (e.g., the colorimetric reagent(s) 104 alone or dry powder blends including the colorimetric reagent 104) is illustrated in FIG. 4.

As further illustrated in FIG. 6A, the breakable ampule 600 can be formed from plastic or glass or flexible polymer pre-filled with the colorimetric reagent 104. FIG. 6B illustrates a broken/snapped/squashed ampule 602 that releases the colorimetric reagent(s) 104 contained therein.

The above examples are effective methods to safely package a solvent and a colorimetric reagent(s), as described in preceding sections of this disclosure. Allowing mass production, packaging, shipping and transport and carriage by end user prior to use.

### Presumptive Colorimetric Reagent

Nitroaromatics—The presumptive colorimetric reagent produces a known visual color indication in the presence of GSR and ER. In particular a known colorimetric indication for the presence of the pre- and post-blast Nitroaromatic ions, is produced by reacting the preceding species with any combination of colorimetric reagents including, by way of example and without limitation, sodium silicate, sodium teraborate, hydroxide salts, N,N,dimethyl-1-naphthylamine, N-1-naphthyl-ethylene-diamine dihydrochloride, or sodium borohydride. In accordance with embodiments of the disclosure, and without limitation, in one embodiment, a suitable presumptive dry colorimetric reagent is sodium tetraborate.

Nitrates, Nitrites, Nitroamines and Nitroesters—The presumptive colorimetric reagent produces a known visual color indication in the presence of GSR and ER. In particular a known colorimetric indication for the presence of the pre- and post-blast Nitrates, Nitrites, Nitroamines and Nitroesters ions, is produced by reacting the preceding species with any combination of (i-iv) below, by way of example and without limitation:
(i) Aromatic amines—by way of example and without limitation, it may be selected from sulfanilamide, sulfanilic acid, 4-Nitroaniline, 3-Nitroaniline, 2-Nitroaniline, 2-Fluoro-5-nitroaniline, 3-Aminobenzenesulfonic acid, 2-Aminobenzenesulfonic acid, 2,4-Diaminobenzenesulfonic acid, 4-Aminobenzoic acid, 3-Aminobenzoic acid, 4-Aminophthalic acid, Methyl 4-aminobenzoate, 3-Fluoro-4-aminobenzoic acid, 2-Aminobenzoic acid, 2-Amino-3-fluorobenzoic acid, 2-Amino-4-fluorobenzoic acid, 2-Amino-5-fluorobenzoic acid, 2-Amino-6-fluorobenzoic acid, 2-Amino-4,5-difluorobenzoic acid, 2-Amino-3-methylbenzoic acid, 2-Amino-5-methylbenzoic acid, 2-Amino-6-methylbenzoic acid, or naphthols. In one embodiment, a suitable presumptive dry aromatic amine reagent is sulfanilamide.
(ii) Acidic or alkaline catalyst—by way of example and without limitation, it may be selected from, oxalic acid, sodium hydrogen sulfate, citric acid, toluene sulfonic acid, sulfamic acid, sulfanilic acid, 3-Aminobenzenesulfonic acid, 2-Aminobenzenesulfonic acid, 2,4-Diaminobenzenesulfonic acid, 4-Aminobenzoic acid, 3-Aminobenzoic acid, 4-Aminophthalic acid, 3-Fluoro-4-aminobenzoic acid, 2-Aminobenzoic acid, 2-Amino-3-fluorobenzoic acid, 2-Amino-4-fluorobenzoic acid, 2-Amino-5-fluorobenzoic acid, 2-Amino-6-fluorobenzoic acid, 2-Amino-4,5-difluorobenzoic acid, 2-Amino-3-methylbenzoic acid, 2-Amino-5-methylbenzoic acid, 2-Amino-6-methylbenzoic acid, benzoic acid, sodium tetra borate, cationic salts of bicarbonate, carbonate, sulphate or the like. In one embodiment, a suitable presumptive dry catalyst reagent is citric acid.
(iii) Azo coupling compound—by way of example, and without limitation, it may be selected from N,N,dimethyl-1-naphthylamine, N-1-naphthyl-ethylene-diamine dihydrochloride, 8-amino-1-naphthol-5-sulphonic acid, or 1-dimethylamino naphthalene-5-sulphonic acid. In one embodiment, a suitable presumptive dry azo coupling reagent is N-1-naphthyl-ethylene-diamine dihydrochloride.
(iv) Reducing compound—by way of example and without limitation may be selected from zinc powders, iron powders, copper powders, or aluminum powders. In accordance with embodiments of the disclosure, and without limitation, in one embodiment, a suitable presumptive dry reducing reagent is zinc dust.

Oxidizers—The presumptive colorimetric reagent produces a known visual color indication in the presence of GSR and ER. In particular a known colorimetric indication for the presence of the pre- and post-blast Oxidizing species, is produced by reacting the preceding species with any combination of, by way of example and without limitation, methylene blue, potassium iodide starch, N,N′-diethyl-p-phenylenediamine sulfate, N,N,dimethyl-1-naphthylamine, N-1-naphthyl-ethylene-diamine dihydrochloride, alpha-naphthol, sulfanilic acid, copper sulphate, iron sulphate, ferricyanide/cyanate salts. In one embodiment, a suitable presumptive dry colorimetric reagent is methylene blue.

Metals—The presumptive colorimetric reagent produces a known visual color indication in the presence of GSR and ER. In particular a known colorimetric indication for the presence of the pre- and post-blast metal particulate, is produced by reacting the preceding species with any combination of, by way of example and without limitation, Sodium Rhodizonate, rubeanic acid, dithizone, alizarin red s, salicylaldoxime, dithiooxamide, 2,9-dimethyl-4,7-diphenyl-1,10-phenanthroline, zinc dibenzyldithiocarbamate, oxalydihydrazide-[bis(2-hydroxyethyl) dithiocarbamate, Neocuproine, Bathocuproine, nickel diethyldithio phosphate, Oxalyldihydrazide, Trimethyl arsonium iodide, 2-Carboxy-2′-hydroxy-5′-sulfoformazylbenzene and its salts, Chlorindazone DS. In one embodiment, a suitable presumptive dry colorimetric reagent is 2-Carboxy-2′-hydroxy-5′-sulfoformazylbenzene and its salts.

### Solvent

In one embodiment, a suitable absorbent material is pre-wetted with a solvent (e.g., a cotton swab or wipe). This modality allows excellent residue dissolution and pickup from surfaces, does not destroy surface coatings or forensic residue, may be applied to human skin and clothing fabrics and has no hazardous chemical features. In an embodiment, a suitable solvent may be selected from aqueous or non-aqueous species. In one embodiment, a suitable is water.

In another embodiment, a suitable solvent is dimethyl sulfoxide.

In another embodiment, a suitable solvent is isopropyl alcohol.

### Packaging

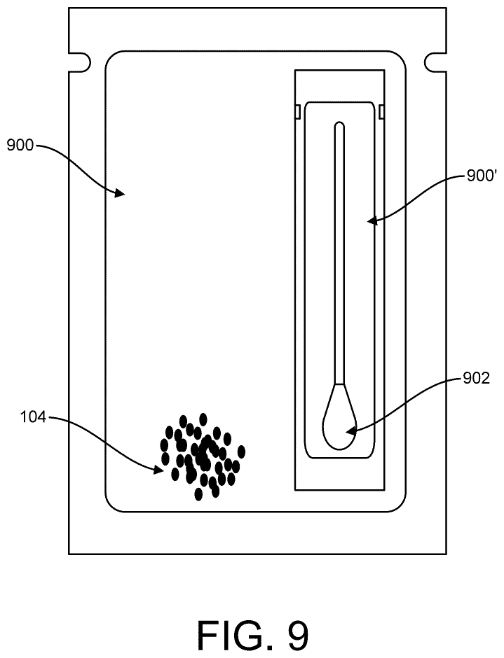

In accordance with embodiments of the disclosure, and without limitation, a kit is provided as illustrated in FIG. 9. In one embodiment, the kit can include the colorimetric reagent(s) 104 and pre-wetted sample collection swab 902 packaged in a moisture and UV resistant package. As shown, the colorimetric reagent(s) can be sealed within a first sachet 900, while the pre-wetted swab can be packaged within a second sachet 900′. Preferably the packaging would be a tare open, form, fill and seal sachet. The sachet would be constructed from commercially available Paper/PET12 um/AL7 um/PE50 product, which is an extremely cheap, mass produced material.

### Use of Kit

In one embodiment, the kit is carried in a pocket, belt case, glove box, brief case, etc. and where a suspect residue is observed, both the pre-wetted swab and color reagent device are removed from the packaging, held in the hand of the operator. The pre-wetted absorbent material is wiped across the suspect surface for approximately 10 seconds, to collect sufficient GSR and/or ER residue. To complete the process, the sample absorbent material is transferred to and inserted into or onto the colorimetric device, with or without agitation, for a period of time, for example 10 seconds. The mixture of the pre-wetted absorbent material solvent, the collected sample and the colorimetric reagent thus facilitates a presumptive colorimetric indication for the presence of GSR and/or ER.

### Color Indications

In accordance with embodiments of the disclosure and without limitation, example results from using the kit are provided below:
Nitroaromatics: (Control—Negative) white—no color change. (Positive Indication) rapid change from white to Purple/Pink/Red.
Nitrates, Nitrites, Nitroesters, Nitroamines: (Control—Negative) white—no color change. (Positive Indication) rapid change from white to Purple/Pink/Red.
Oxidisers: (Control—Negative) blue—no color change or white. (Positive Indication) rapid change from blue to Purple/Pink or Brown/Black.
Metals: (Control—Negative) Purple—white. (Positive Indication) rapid change from Purple to blue.

### General Definitions

The following definitions are included for the purpose of understanding the present subject matter and for constructing the appended patent claims. The abbreviations used herein have their conventional meanings within the chemical and biological arts.

While various embodiments and aspects of embodiments of the disclosure are shown and described herein, it will be obvious to those skilled in the art that such embodiments and aspects are provided by way of example only. Numerous variations, changes, and substitutions will now occur to those skilled in the art without departing from the disclosed embodiments. It should be understood that various alternatives to the embodiments described herein may be employed.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of the disclosed embodiments. The following definitions are provided to facilitate understanding of certain terms used frequently herein and are not meant to limit the scope of the present disclosure.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed embodiments.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by embodiments of the disclosure. For example, “0.2-5 mg” is a disclosure of 0.2 mg, 0.3 mg, 0.4 mg, 0.5 mg, 0.6 mg etc. up to and including 5.0 mg.

Approximating language, as used herein throughout the specification and claims, may be applied to modify any quantitative representation that could permissibly vary without resulting in a change in the basic function to which it is related. “Approximately,” “substantially, or “about” can include numbers that fall within a range of 1%, or in some embodiments within a range of 5% of a number, or in some embodiments within a range of 10% of a number in either direction (greater than or less than the number) unless otherwise stated or otherwise evident from the context (except where such number would impermissibly exceed 100% of a possible value). Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

As used in the description herein and throughout the claims that follow, the meaning of “a,” “an,” and “the” includes plural reference unless the context clearly dictates otherwise.

The term “small molecule” includes its ordinary and customary meaning and may further refer to broadly as an organic, inorganic or organometallic compound with a low molecular weight compound (e.g., a molecular weight of less than about 2,000 Da or less than about 1,000 Da). The small molecule may have a molecular weight of less than about 2,000 Da, a molecular weight of less than about 1,500 Da, a molecular weight of less than about 1,000 Da, a molecular weight of less than about 900 Da, a molecular weight of less than about 800 Da, a molecular weight of less than about 700 Da, a molecular weight of less than about 600 Da, a molecular weight of less than about 500 Da, a molecular weight of less than about 400 Da, a molecular weight of less than about 300 Da, a molecular weight of less than about 200 Da, a molecular weight of less than about 100 Da, or a molecular weight of less than about 50 Da.

Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecules can be synthetically engineered to consist of a fragment, or small portion, or a longer amino acid chain to fill a binding pocket of an enzyme. Typically small molecules are less than one kilodalton.

As used herein, the term “stereoisomers” includes its ordinary and customary meaning and includes compounds made up of the same atoms having the same bond order but having different three-dimensional arrangements of atoms that are not interchangeable. The three-dimensional structures are called configurations. As used herein, the term “enantiomers” refers to two stereoisomers that are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

By “alteration” is meant a change (increase or decrease) in the presence of the small molecule, e.g., Fentanyl analogue, as detected by standard methods known in the art such as those described herein. As used herein, an alteration includes a 10% or more change in presence, preferably a 25% change, more preferably a 40% change, and most preferably a 50% or greater change in the presence.

As used herein an “alteration” also includes a 2-fold or more change, for example, 5-fold, 10-fold, 20-fold, 30-fold, 40-fold, 50-fold, 100-fold, 500-fold, 1000-fold or more.

The term “sample” includes its ordinary and customary meaning and includes a biological sample obtained for the purpose of evaluation in vitro. In embodiments, the sample may comprise a body fluid. In some embodiments, the body fluid includes, but is not limited to, whole blood, plasma, serum, lymph, breast milk, saliva, mucous, semen, cellular extracts, inflammatory fluids, cerebrospinal fluid, vitreous humor, tears, vitreous, aqueous humor, or urine obtained from the subject. In some aspects, the sample is a composite panel of two or more body fluids. In exemplary aspects, the sample comprises blood or a fraction thereof (e.g., plasma, serum, or a fraction obtained via leukapheresis).

A “control” sample or value refers to a sample that serves as a reference, usually a known reference, for comparison to a test sample. For example, a test sample can be taken from a test subject, and compared to samples from known conditions, e.g., a subject (or subjects) that does not have the disease (a negative or normal control), or a subject (or subjects) who does have the disease (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

For example, the method can further be a high throughput method. In aspects, a high throughput method may refer to an assay which provides for multiple candidate agents, samples or test compound to be screened simultaneously. As further described below, examples of such assays may include the use of microtiter plates that are especially convenient because a large number of assays can be carried out simultaneously, using small amounts of reagents and samples. The methods are easily carried out in a multiwell format including, but not limited to, 96-well and 384-well formats and automated.

The term “gun shot residue” includes its ordinary and customary meaning and includes residue from discharged weapon thereof. The term “equivalent to about . . . of GSR” is intended to relate to a specified volume, concentration, or amount of GSR provided by a volume, concentration, or mass.

The term “explosive residue” includes its ordinary and customary meaning and includes residue from discharged weapon or explosive device thereof. The term “equivalent to about . . . of ER” is intended to relate to a specified volume, concentration, or amount of ER provided by a volume, concentration, or mass.

As used herein, “salts” or “salt form” or “pharmaceutically accepted salts” may include base addition salts (formed with free carboxyl or other anionic groups) which are derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts are formed as acid addition salts with any free cationic groups and generally are formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like. Salts of the disclosure may include amine salts formed by the protonation of an amino group with inorganic acids such as hydrochloric acid, hydrobromic acid, hydroiodic acid, sulfuric acid, phosphoric acid, and the like. Salts of the disclosure also include amine salts formed by the protonation of an amino group with suitable organic acids, such as p-toluenesulfonic acid, acetic acid, and the like. Additional excipients which are contemplated for use in the practice of the present disclosure are those available to those of ordinary skill in the art, for example, those found in the United States Pharmacopoeia Vol. XXII and National Formulary Vol. XVII, U.S. Pharmacopoeia Convention, Inc., Rockville, Md. (1989), the relevant contents of which is incorporated herein by reference.

### EXAMPLES

The following examples illustrate certain specific embodiments of the disclosure and are not meant to limit the scope of the disclosed embodiments.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1

Metal detection swab: In accordance with embodiments of the present disclosure and without limitation, a presumptive kit for the detection and identification of metal particulate GSR and or ER is produced by pre-wetting a cotton swab with 0.05 to 0.2 mL “Solvent 1”. Individually packaging said swab into a hermetically form fill sealed Paper/PET12 um/AL7 um/PE50 sachet.

In accordance with embodiments of the present disclosure and without limitation, said “Solvent 1” is composed of a saturated sodium tetraborate/water solution.

Metal detection colorimetric device: In accordance with embodiments of the present disclosure and without limitation, a presumptive kit for the detection and identification of metal particulate GSR and or ER is produced by impregnating a bibulous carrier with an alcoholic solution of 2-Carboxy-2′-hydroxy-5′-sulfoformazylbenzene and its salts. Individually packaging said bibulous carrier into a hermetically form fill sealed Paper/PET12 um/AL7 um/PE50 sachet.

In accordance with embodiments of the present disclosure and without limitation, the above colorimetric reagent solution may be produced by dissolving 0.2 grams of 2-Carboxy-2′-hydroxy-5′-sulfoformazylbenzene and its salts in 70 mL of ethanol.

In accordance with embodiments of the present disclosure and without limitation, the bibulous carrier is Whatman chromatography paper #1.

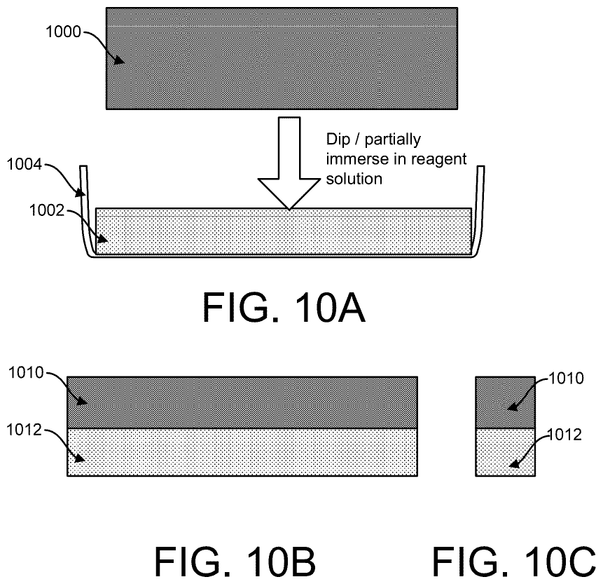

In accordance with embodiments of the present disclosure and without limitation a suitable method of applying the colorimetric reagent to the chromatography paper is a “dip-n-dry” or printing process. A dip-n-dry process is illustrated in FIGS. 10(a)-10(c). As shown, a chromatography paper 1000 (e.g. Whatman #1 CP), is partially dipped into a reagent solution 1002 held in a dipping tray 1004. The reagent solution 1002 wicks and absorbs into the chromatography paper 1000. The wetted chromatography paper 100 is removed from the tray 1004 for subsequent drying by hot air stream. As further illustrated in FIG. 10(b), after dipping and drying, the chromatography paper 1000 include a portion that is impregnated with dried reagent 1002. The chromatography paper can be subsequently cut to a desired use shape (e.g., a strip).

### Example 2

Nitroaromatics—In accordance with embodiments of the present disclosure and without limitation, a presumptive kit for the detection and identification of nitroaromatics in GSR and or ER is produced by applying sodium tetraborate to and coating a solid support substrate. In certain embodiments, the coated solid support can be individually packaged. For example, the coated solid support substrate can be individually packaged within a hermetically sealed sachet (e.g., a form fill sealed Paper/PET12 um/AL7 um/PE50 sachet).

In accordance with embodiments of the present disclosure and without limitation, the coating process is flexographic printing, and the printing is achieved by containing said solid support with reagent solution, drying the coated solid support and guillotining to any desired shape. The final article is packaged as described above.

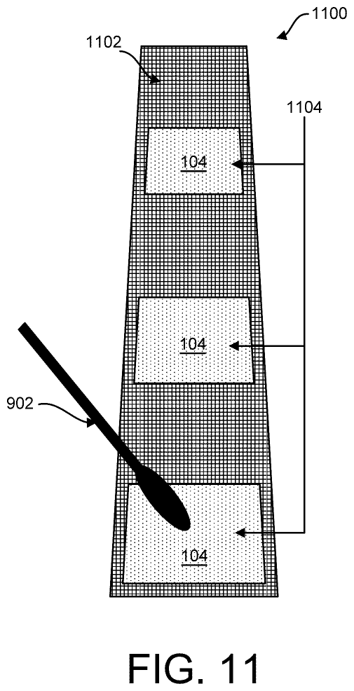

FIG. 11 illustrates an exemplary embodiment of a coated solid support substrate according to embodiments of the disclosure in the form of a printed dry reagent test strip 1100. The dry reagent powder mix 104 is added to and mixed with a solvent and suitable gelling agent, to produce a homogenous suspension and printed to any solid support substrate 1102. The dried article (e.g. test strip 1100) may have one or many reagent dye test zones 1104 to detect and presumptively identify one or more unknown compounds.

In accordance with embodiments of the present disclosure and without limitation, the solid support substrate is 300 gsm card or synthetic plastic paper.

In accordance with embodiments of the present disclosure and without limitation, the reagent solution or “printing ink” is produced by dissolving 1 gram of sodium tetraborate in 100 mL sodium silicate.

Nitroaromatic detection swab: In accordance with embodiments of the present disclosure and without limitation, a presumptive kit for the detection and identification of metal particulate GSR and or ER is produced by pre-wetting a cotton swab with 0.05 to 0.2 mL “Solvent 2”. The swab can be individually packaged into a hermetically sealed sachet (e.g., a form fill sealed Paper/PET12 um/AL7 um/PE50 sachet).

In accordance with embodiments of the present disclosure and without limitation, the “Solvent 2” is composed of dimethyl sulfoxide (DMSO) and isopropyl alcohol (75:25).

### Example 3

Nitrates, Nitrites, Nitroamines and Nitroesters—In accordance with embodiments of the present disclosure and without limitation, a presumptive kit for the detection and identification of nitroaromatics in GSR and or ER is produced by applying any combination of sulfanilamide, sulfanilic acid, citric acid, N-1-naphthyl-ethylene-diamine dihydrochloride to and coating a solid support substrate. Individually packaging said coated solid support into a hermetically form fill sealed Paper/PET12 um/AL7 um/PE50 sachet.

In accordance with embodiments of the present disclosure and without limitation, the coating process is flexographic printing, and the printing is achieved by containing said solid support with reagent solution, drying the coated solid support and guillotining to any desired shape. The final article is packaged as described above.

In accordance with embodiments of the present disclosure and without limitation, the solid support substrate is 300 gsm card or synthetic plastic paper.

In accordance with embodiments of the present disclosure and without limitation, the reagent solution or “printing ink” is produced by dissolving: (i) 1 gram of sulfanilamide, (ii) 1 gram of sulfanilic acid, (iii) 1 gram of N-1-naphthyl-ethylene-diamine dihydrochloride, (iv) 50 grams of citric acid in 500 mL of aqueous alcoholic solvent.

In accordance with embodiments of the present disclosure and without limitation, the aqueous alcoholic solvent may be prepared in any ration of water: isopropyl alcohol. A suitable ratio is 30:70.

Nitrates, Nitrites, Nitroamines and Nitroesters detection swab: In accordance with embodiments of the present disclosure and without limitation, a presumptive kit for the detection and identification of metal particulate GSR and or ER is produced by pre-wetting a cotton swab with 0.05 to 0.2 mL “Solvent 3”. In an embodiment, the pre-wetted swab can be individually packaged into a hermetically sealed sachet (e.g., a form fill sealed Paper/PET12 um/AL7 um/PE50 sachet).

In accordance with embodiments of the present disclosure and without limitation, said “Solvent 3” is composed of dimethylsulfoxide (DMSO) and isopropyl alcohol (75:25).

### Example 4

Oxidizers—In accordance with embodiments of the present disclosure and without limitation, a presumptive kit for the detection and identification of oxidizers in GSR and or ER is produced by applying methylene blue to and coating a solid support substrate. In an embodiment, the solid support substrate can be individually packaged. As an example, the solid support substrate can be individually packaged within a hermetically sealed sachet (e.g., a form fill sealed Paper/PET12 um/AL7 um/PE50 sachet).

In accordance with embodiments of the present disclosure and without limitation, the coating process is flexographic printing, and the printing is achieved by containing said solid support with reagent solution, drying the coated solid support and guillotining to any desired shape. The final article is packaged as described above.

In accordance with embodiments of the present disclosure and without limitation, the solid support substrate is 300 gsm card or synthetic plastic paper.

In accordance with embodiments of the present disclosure and without limitation, the reagent solution or “printing ink” is produced by dissolving: 0.2 gram of methylene blue in 100 mL of ethanol solvent.

Oxidizers detection swab: In accordance with embodiments of the present disclosure and without limitation, a presumptive kit for the detection and identification of metal particulate GSR and or ER is produced by pre-wetting a cotton swab with 0.05 to 0.2 mL “Solvent 4”. Individually packaging said swab into a hermetically form fill sealed Paper/PET12 um/AL7 um/PE50 sachet.

In accordance with embodiments of the present disclosure and without limitation, said “Solvent 4” is composed of water.

While the disclosed test kit of embodiments of the present disclosure has been shown and described in detail, it is obvious that such embodiments are not to be considered as limited to the exact form disclosed, and that changes in detail and construction may be made therein within the scope of the disclosure without departing from the spirit thereof.

### REFERENCES

Each of the references listed below are incorporated by reference in their entirety.
U.S. Pat. No. 6,613,576—Rodacy, P. and Walker, P. Field kit and method for testing for the presence of gunshot residue. 2002.
BR 102014004252—De Almeida, et al., Composition for identification of firearm residues, firearm preparation process and method for detecting lead residues. 2014
US 2017/0153180—Basu, Partha, et al. Compositions, methods and devices thereof for fluorescent analysis of gunshot residue. 2017.
U.S. Pat. No. 6,613,576—Rodacy, P. et al. Field kit and method for testing for the presence of gunshot residue. 2003.
U.S. Pat. No. 7,410,612—Carrington, J. Gunpowder particle test kit. 2008.
U.S. Pat. No. 7,499,808—Sinha, S. Method and system for characterizing nanoparticles in a gun-shot residue. 2009.
U.S. Pat. No. 9,880,092—Foy, D. et al. Visual indication test kit. 2018.
U.S. Pat. No. 10,260,999—Glattstein, A. and Glattstein, B. Sampling device. 2019.
WO 2010/105326—Webber, I. et al. Process for producing luminescent ammunition and detecting gun shot residues. 2010.
U.S. Pat. No. 3,748,096—Schmitt, D., et al. Indicator for the detection of copper ions, 1970.
U.S. Pat. No. 5,620,658—Jaunakais, I. Colorimetric test strip, 1995.
[1] www.firearmsid.com/A_distanceGSR.htm
[2] www.sciencedirect.com/topics/chemistry/gunpowder
Charles, S., et. al., Interpol review of gunshot residue 2016-2019. Forensic Science International. https://www.sciencedirect.com/science/article/pii/S2589871X20300115

### Other Embodiments

While embodiments of the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While certain embodiments have been particularly shown and described herein, it will be understood by those skilled in the art that various changes in form and details may be made therein without departing from the scope of the disclosed embodiments encompassed by the appended claims.

What is claimed:

1. A portable detection kit for identifying the presence of gunshot residue (GR) and explosive residue (ER), wherein the kit comprises:
a colorimetric reagent(s),
a solvent(s), and
a swab(s),
wherein the colorimetric reagent(s) and solvents(s) and a swab(s) are be provided in hermetically sealed light, air and moisture proof packaging.

2. The portable detection kit as in claim 1, wherein the GSR and ER comprise any discharge from weapons and explosives.

3. The portable detection kit as in claim 2, wherein the GSR or ER are present as liquids, gels or solid residues that are pure or admixed with clandestine cutting or diluting agents.

4. The portable detection kit of claim 1, wherein the colorimetric reagent(s) in combination with the solvent(s) are configured to undergo physico-chemical interaction with GSR and ER, producing colored reaction product(s), resulting in a visible color change.

5. The portable detection kit of claim 1, wherein the GSR or ER is Metal Particulate (MP) and the swab is a pre-wetted cotton swab for the collection of MP.

6. The portable detection kit of claim 1, wherein the swab is pre-wetted with a solution comprising the solvent(s) and the colorimetric reagent(s).

7. The portable detection kit of claim 6, wherein the solution is a volume of 0.05 to 0.2 mL of a saturated sodium tetraborate/water solution and the pre-wetted swab is individually packaged into a hermetically sealed sachet.

8. The portable detection kit of claim 1, wherein the colorimetric reagent(s) is selected from the group consisting of: Sodium Rhodizonate, rubeanic acid, dithizone, alizarin red s, salicylaldoxime, dithiooxamide, 2,9-dimethyl-4,7-diphenyl-1,10-phenanthroline, zinc dibenzyldithiocarbamate (ZDBT). ZDBT, oxalydihydrazide-[bis(2-hydroxyethyl) dithiocarbamate, Neocuproine, Bathocuproine, nickel diethyldithio phosphate, Oxalyldihydrazide, Trimethyl arsonium iodide, 2-Carboxy-2′-hydroxy-5′-sulfoformazylbenzene and its salts, Chlorindazone DS.

9. The portable detection kit of claim 1, wherein the colorimetric reagent(s) is selected from the group consisting of 2-Carboxy-2′-hydroxy-5′-sulfoformazylbenzene and its salts.

10. The portable detection kit of claim 1, further comprising a bibulous carrier including a solution of the colorimetric reagent(s) and the solvent(s) absorbed therein.

11. The portable detection kit of claim 10, wherein the solution comprises 0.2 grams of 2-Carboxy-2′-hydroxy-5′-sulfoformazylbenzene or its salts in 70 mL of ethanol.

12. The portable detection kit of claim 10, wherein the bibulous carrier is chromatography filter paper.

13. The portable detection kit of claim 1, wherein the GSR or ER is a nitroaromatic (NA) and the swab is pre-wetted for the collection of NA.

14. The portable detection kit of claim 13, wherein swab is pre-wetted with a solution comprising the solvent and the colorimetric reagent(s).

15. The portable detection kit of claim 14, wherein the solution is a volume a 75% dimethylsulfoxide (DMSO) and 25% isopropyl alcohol solution and the pre-wetted swab is individually packaged into a hermetically sealed sachet.

16. The portable detection kit of claim 1, wherein the colorimetric reagent(s) is selected from the group consisting of sodium silicate, sodium teraborate, hydroxide salts, N,N,dimethyl-1-naphthylamine, N-1-naphthyl-ethylene-diamine dihydrochloride, and sodium borohydride.

17. The portable detection kit of claim 1, further comprising a solid support substrate including a solution of the colorimetric reagent(s) and the solvent(s) applied thereon.

18. The portable detection kit of claim 17, wherein the solution comprises 1 gram of sodium tetraborate in 100 mL sodium silicate.

19. The portable detection kit of claim 17, wherein the solid support substrate is 300 gsm card or synthetic paper.

20. The portable detection kit of claim 1, wherein the GSR or ER is at least one of Nitrates, Nitrites, Nitroamines and Nitroesters (NNNN) and the swab is a pre-wetted cotton swab for the collection of NNNN.

21. The portable detection kit of claim 20, wherein the swab is pre-wetted with the solvent(s).

22. The portable detection kit of claim 21, wherein the solvent(s) is a volume of 75% DMSO and 25% isopropyl alcohol and the pre-wetted swab is individually packaged into a hermetically sealed sachet.

23. The portable detection kit of claim 1, wherein the colorimetric reagent(s) is selected from the group consisting of: sulfanilamide, sulfanilic acid, 4-Nitroaniline, 3-Nitroaniline, 2-Nitroaniline, 2-Fluoro-5-nitroaniline, 3-Aminobenzenesulfonic acid, 2-Aminobenzenesulfonic acid, 2,4-Diaminobenzenesulfonic acid, 4-Aminobenzoic acid, 3-Aminobenzoic acid, 4-Aminophthalic acid, Methyl 4-aminobenzoate, 3-Fluoro-4-aminobenzoic acid, 2-Aminobenzoic acid, 2-Amino-3-fluorobenzoic acid, 2-Amino-4-fluorobenzoic acid, 2-Amino-5-fluorobenzoic acid, 2-Amino-6-fluorobenzoic acid, 2-Amino-4,5-difluorobenzoic acid, 2-Amino-3-methylbenzoic acid, 2-Amino-5-methylbenzoic acid, 2-Amino-6-methylbenzoic acid, naphthols, oxalic acid, sodium hydrogen sulfate, citric acid, toluene sulfonic acid, sulfamic acid, sulfanilic acid, 3-Aminobenzenesulfonic acid, 2-Aminobenzenesulfonic acid, 2,4-Diaminobenzenesulfonic acid, 4-Aminobenzoic acid, 3-Aminobenzoic acid, 4-Aminophthalic acid, 3-Fluoro-4-aminobenzoic acid, 2-Aminobenzoic acid, 2-Amino-3-fluorobenzoic acid, 2-Amino-4-fluorobenzoic acid, 2-Amino-5-fluorobenzoic acid, 2-Amino-6-fluorobenzoic acid, 2-Amino-4,5-difluorobenzoic acid, 2-Amino-3-methylbenzoic acid, 2-Amino-5-methylbenzoic acid, 2-Amino-6-methylbenzoic acid, benzoic acid, sodium tetra borate, cationic salts of bicarbonate, carbonate, sulphate and the like, N,N,dimethyl-1-naphthylamine, N-1-naphthyl-ethylene-diamine dihydrochloride, 8-amino-1-naphthol-5-sulphonic acid, and 1-dimethylamino naphthalene-5-sulphonic acid, zinc powder, iron powder, copper powder, and aluminum powder.

24. The portable detection kit of claim 1, wherein the colorimetric reagent(s) is provided a solution with the solvent(s), wherein the solution comprises (i) 1 gram of sulfanilamide, (ii) 1 gram of sulfanilic acid, (iii) 1 gram of N-1-naphthyl-ethylene-diamine dihydrochloride, (iv) 50 grams of citric acid in 500 mL of aqueous alcohol solvent.

25. The portable detection kit of claim 24, wherein the aqueous alcoholic solvent comprises water and isopropyl alcohol in a predetermined ratio.

26. The portable detection kit of claim 25, further comprising a solid support substrate including the solution applied thereon.

27. The portable detection kit of claim 26, wherein the solid support substrate is 300 gsm card or synthetic paper and packaged into a hermetically sealed sachet.

28. The portable detection kit of claim 1 wherein the GSR or ER is an oxidizer (OX) and the swab is a pre-wetted cotton swab for the collection of OX.

29. The portable detection kit of claim 28, wherein the swab is pre-wetted with a solution comprising the solvent(s) and the colorimetric reagent(s).

30. The portable detection kit of claim 29, wherein the solvent(s) is water.

31. The portable detection kit of claim 1, wherein the colorimetric reagent(s) is selected from the group consisting of: methylene blue, potassium iodide starch, N,N′-diethyl-p-phenylenediamine sulfate, N,N,dimethyl-1-naphthylamine, N-1-naphthyl-ethylene-diamine dihydrochloride, alpha-naphthol, sulfanilic acid, copper sulphate, iron sulphate, and ferricyanide/cyanate salts.

32. The portable detection kit of claim 1, wherein the colorimetric reagent(s) is methylene blue.

33. The portable detection kit of claim 31, further comprising a solid support substrate including a solution of the colorimetric reagent(s) and solvent(s) applied thereon.

34. The portable detection kit of claim 33, wherein the solution comprises 0.2 gram of methylene blue in 100 mL of ethanol solvent.

35. The portable detection kit of claim 34, wherein the solid support substrate is 300 gsm card or synthetic paper and the solid support substrate is packaged into a hermetically sealed sachet.

36. The portable detection kit of claim 1, wherein the solvent(s) are configured to dissolve the colorimetric reagent(s), GSR, and ER.

37. The portable detection kit of claim 1, wherein the solvent(s) comprise aqueous and non-aqueous solvents.

38. The portable detection kit of claim 37, wherein the solvent(s), comprises at least one of water, dimethyl sulfoxide, or alcohols.

39. The portable detection kit of claim 38, wherein the swab(s) is a cotton swab that is pre-wetted with the solvent(s), and individually packaged into a hermetically sealed sachet.

40. The portable detection kit of claim 1, wherein the colorimetric reagent(s) comprises a mixture of at least two dry powder reagents.

41. The portable detection kit of claim 40, wherein the mixed dry powder reagents exhibit a homogenized micronized size within the range from about 0.1 micron to about 30 micron.

42. The portable detection kit of claim 1, further comprising a sealed sachet containing about 0.1 grams to about 1 grams of the colorimetric reagent(s).

43. The portable detection kit of claim 1, further comprising a solid support substrate including a solution of the colorimetric reagent(s) applied thereon.

44. The portable detection kit of claim 1, further comprising a sealed sachet containing the colorimetric reagent(s), the solvent(s), and the swab(s).

45. The portable detection kit of claim 44, wherein the sachet is formed from paper, PET12 um, AL7 um, or PE50.

46. A method of detecting gunshot residue (GSR) or explosive residue (ER), comprising:
providing the portable detection kit of claim 1, including hermetically sealed sachets containing the swab pre-wetted with a water solvent and dry powder of the colorimetric reagent(s);
locating a residue or surface suspected to include GSR and/or ER; and
opening both the sealed sachets containing the colorimetric reagent(s) and the pre-wetted swab.

47. The method of claim 46, further comprising:
removing the pre-wetted swab from its sachet,
applying a swab tip to the suspect residue and/or surface, and
rubbing the swab tip on the residue and/or surface to thereby dissolve and collect GSR and/or ER, yielding a sampled swab.

48. The method of claim 47, further comprising contacting the sampled swab with the colorimetric reagent(s) for a period of at least 10 seconds.

49. The method claim 48, further comprising monitoring both the swab tip and colorimetric reagent(s) for development of a presumptive colorimetric indication for the presence of GSR and/or ER.

50. The method of claim 49, wherein presumptive indications are: (metal negative—purple to white)(metals positive—purple to blue), (nitroaromatics (NA) negative—white to white)(NA positive—white to pink/red), (Nitrates, Nitrites, Nitroamines or Nitroesters (NNNN) negative—white to white)(NNNN positive—white to pink/red), (oxidizers (OX) negative—blue to blue or white)(OX positive—blue to pink/purple).

51. A method of detecting gunshot residue (GSR) or explosive residue (ER), comprising providing the portable detection kit of claim 1, wherein the swab(s) comprises a hollow shaft and a tip containing a fiber matrix and the colorimetric reagent(s) comprise dry powder reagent(s) embedded into the tip fiber matrix and wherein a water solvent is contained within the hollow shaft, and wherein the kit comprises the swab(s) and the colorimetric reagent(s) hermetically sealed within respective portions of a sachet.

52. The method of claim 51, further comprising:
locating a residue or surface suspected to include GSR and/or ER; and
opening the hermetically sealed sachet containing the swab(s).

53. The method of claim 52, further comprising:
removing the swab(s) from its sachet;
breaking the hollow shaft of the swab to releasing the water solvent into the tip of the swab;
applying the swab tip to the suspect residue and/or surface; and
rubbing the swab tip on the residue and/or surface to dissolve and collect GSR and/or ER, yielding a sampled swab.

54. The method of claim 53, further comprising monitoring both the swab tip and the rubbed surface to detect an indication comprising one of: (A) a purple-pink-red color formation representing positive presumptive colorimetric indication for the presence of GSR and/or ER or (B) a white swab tip/no color change representing a negative result.

55. A method of detecting gunshot residue (GSR) or explosive residue (ER), comprising:
providing the portable detection kit of claim 1, wherein the swab(s) comprises a swab that is pre-wetted with a water solvent and is sealed in a sachet, and wherein the colorimetric reagent(s) comprise one of dry powder reagents sealed in a sachet, a sheet sealed in a sachet, dry powder reagent(s) sealed in a breakable ampule or blister pack, a tablet or pellet sealed in a sachet;
locating a residue or surface suspected to include GSR and/or ER; and
opening the sachet containing the pre-wetted swab and the sachet, ampule, or blister pack containing the colorimetric reagent(s).

56. The method of claim 55, further comprising:
removing the pre-wetted water swab from its sachet;
applying a swab tip to the suspect residue and/or surface; and
rubbing the swab tip on the residue and/or surface to thereby dissolve and collect the GSR and/or ER, yielding a sampled swab.

57. The method of claim 56, further comprising:
inserting the sampled swab into the opened sachet, blister pack, or ampoule;
contacting the sampled swab with the powder, sheet, tablet or pellet containing the colorimetric reagent(s), for a period of at least 10 seconds; and
waiting for the development of a positive colorimetric reaction.

58. The method of claim 57, further comprising observation of both the swab tip and the colorimetric reagent(s) to detect an indication comprising one of: (A) a purple-pink-red color formation representing positive presumptive colorimetric indication for the presence of GSR and/or ER or (B) a white swab tip/no color change representing a negative result.
